(19) United States
(12) Patent Application Publication
(10) Pub. No.: US 2004/0216242 A1
(43) Pub. Date: Nov. 4, 2004

(54) Dyeing composition comprising a diaminopyrazole-type oxidation base a heterocyclic oxidation base and a coupling agent

(76) Inventors: Sylvain Kravtchenko, Asnieres (FR); Alain Lagrange, Coupvray (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/490,861

(22) PCT Filed: Sep. 27, 2002
(86) PCT No.: PCT/FR02/03317

(30) Foreign Application Priority Data
Sep. 28, 2001 (FR) 01/12527

(51) Int. Cl. A61K 7/13

(52) U.S. Cl. 8/405

(57) ABSTRACT

The invention relates to a dye composition comprising an oxidation base of the diaminopyrazole type, a heterocyclic oxidation base and a coupler.

The invention also relates to the use of this composition for dyeing keratin fibers and also to the dyeing process using this composition.

The invention relates to a dye composition comprising an oxidation base of the diaminopyrazole type, a heterocyclic oxidation base and a coupler. The invention also relates to the use of this composition for dyeing keratin fibers and also to the dyeing process using this composition.

It is known practice to dye keratin fibers and in particular human hair with dye compositions containing oxidation dye precursors, generally known as oxidation bases, such as ortho- or para-phenylene-diamines, ortho- or para-aminophenols and heterocyclic compounds. These oxidation bases are colorless or weakly colored compounds, which, when combined with oxidizing products, may give rise to colored compounds via a process of oxidative condensation.

It is also known that the shades obtained with these oxidation bases may be varied by combining them with couplers or coloration modifiers, the latter being chosen especially from aromatic meta-diamines, meta-aminophenols, meta-diphenols and certain heterocyclic compounds such as indole compounds.

The variety of molecules used as oxidation bases and couplers allows a wide range of colors to be obtained.

The “permanent” coloration obtained by means of these oxidation dyes must moreover satisfy a certain number of requirements. Thus, it must have no toxicological drawbacks, it must allow shades to be obtained in the desired intensity, and it must show good resistance to external agents such as light, bad weather, washing, permanent waving, perspiration and rubbing.

The dyes must also make it possible to cover white hair and, finally, they must be as unselective as possible, i.e. they must produce the smallest possible color differences along the same length of keratin fiber, which is generally differently sensitized (i.e. damaged) between its end and its root.

Dye compositions comprising diaminopyrazole derivatives as oxidation bases are already known. For example, patent application DE 3 843 892 describes dye compositions for dyeing keratin fibers, comprising 4,5-diaminopyrazole derivatives that may be substituted in position 2 with alkyl or hydroxyalkyl radicals. Patent application EP 692 245 describes dye compositions comprising 4,5-diaminopyrazole derivatives combined with particular meta-phenylenediamines. Patent application DE 196 43 059 describes dye compositions combining 4,5-diaminopyrazole derivatives with meta-aminophenol and meta-phenylenediamine couplers. Patent application DE 196 46 609 describes dye compositions combining 4,5-diaminopyrazole derivatives with benzoxazine couplers.

The aim of the present invention is to provide novel dye compositions for dyeing keratin fibers, containing diaminopyrazole derivatives, which do not have the drawbacks of those of the prior art. In particular, the aim of the present invention is to provide dye compositions containing diaminopyrazole derivatives that are relatively unselective and particularly fast, while at the same time being capable of generating intense colorations in varied shades.

This aim is achieved with the present invention, one subject of which is a dye composition comprising, in a suitable dyeing medium:

at least one 4,5-diaminopyrazole oxidation base of formula (I) or the addition salts thereof:

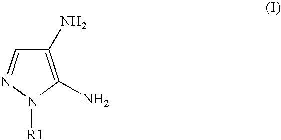

in which R1 is a C₁-C₆ alkyl radical substituted with one or more radicals OR, R being a C₁-C₆ alkyl radical,

at least one second heterocyclic oxidation base, and

at least one coupler.

Another subject of the invention is the use of the composition of the present invention for dyeing keratin fibers, in particular human keratin fibers such as the hair.

A subject of the invention is also a dyeing device and a dyeing process using the composition of the invention.

In the context of the present invention, the term “alkyl” means linear or branched radicals, for example methyl, ethyl, n-propyl, isopropyl, butyl, etc.

According to one particular embodiment, the 4,5-diaminopyrazole oxidation base of formula (I) is such that R1 represents a C₁-C₄ and preferably C₂-C₄ alkyl radical substituted with a radical OR, R being a C₁-C₄ and preferably C₁-C₂ alkyl radical. The 4,5-diaminopyrazole oxidation base of formula (I) is preferably 4,5-diamino-1-(2′-methoxyethyl)pyrazole.

Any heterocyclic oxidation base that is standard in oxidation dyeing, other than the heterocyclic oxidation bases of formula (I), may be used in the dye composition of the present invention. In particular, the heterocyclic oxidation base(s) that is (are) useful in the context of the invention is (are) chosen from pyridine, pyrimidine and pyrazole heterocyclic oxidation bases, and the addition salts thereof.

Among the pyridine oxidation bases that may be mentioned are the compounds described, for example, in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Other pyridine oxidation bases that are useful in the present invention are the 3-aminopyrazolo[1,5-a]pyridine oxidation bases or the addition salts thereof described, for example, in patent application FR 2 801 308. By way of example, mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo-[1,5-a]pyridine-2-carboxylic acid; 2-methoxypyrazolo-[1,5-a]pyrid-3-ylamine; (3-aminopyrazolo[1,5-a]pyrid-7-yl)methanol; 2-(3-aminopyrazolo[1,5-a]pyrid-5-yl)-ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]-ethanol; 2-[(3-aminopyrazolo[1,5-a]pyrid-7-yl)-(2-hydroxyethyl)amino]ethanol; 3-aminopyrazolo-[1,5-a]pyrid-5-ol; 3-aminopyrazolo[1,5-a]pyrid-4-ol; 3-aminopyrazolo[1,5-a]pyrid-6-ol; 3-aminopyrazolo-[1,5-a]pyrid-7-ol and also the addition salts thereof with an acid or with a base.

Among the pyrimidine derivatives that may be mentioned are the compounds described, for example, in German patent DE 2 359 399 or Japanese patents JP 88-169 571; JP 05 163 124; EP 0 770 375 or patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]-pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo-[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid and the tautomeric forms thereof, when a tautomeric equilibrium exists.

Among the pyrazole derivatives that may be mentioned are the compounds described in patents DE 3 843 892 and DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

According to one particular embodiment, the heterocyclic oxidation base that is useful in the composition of the present invention is a 3-aminopyrazolo[1,5-a]pyridine oxidation base. Preferably, the 3-aminopyrazolo[1,5-a]pyridine oxidation base is pyrazolo[1,5-a]pyrid-3-ylamine.

The composition of the present invention comprises at least one coupler. The couplers that are useful may be chosen from meta-phenylenediamines, meta-aminophenols, meta-diphenols, naphthalene-based couplers and heterocyclic couplers, and the addition salts thereof.

Examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene and the addition salts thereof.

According to one particular embodiment, the coupler is a meta-aminophenol.

In the composition of the present invention, the amount of each of the couplers is between 0.001% and 10% and preferably between 0.005% and 6% by weight approximately relative to the total weight of the dye composition.

In addition to the compounds described above, the composition of the present invention may contain one or more additional oxidation bases chosen from the oxidation bases conventionally used in oxidation dyeing, other than the oxidation bases described above. These additional oxidation bases are chosen, for example, from para-phenylenediamines, bis(phenyl)-alkylenediamines, para-aminophenols and ortho-aminophenols, and the addition salts thereof.

Among the para-phenylenediamines that may be mentioned more particularly, for example, are para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-p-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above that are particularly preferred are para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid.

Among the bis(phenyl)alkylenediamines that may be mentioned, for example, are N,N′-bis(β-hydroxyethyl)-N,N′-bis (4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylene-diamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols that may be mentioned, for example, are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof with an acid.

The amount of each of the oxidation bases present in the composition of the invention is generally between 0.001% and 10% and preferably between 0.005% and 6% by weight approximately relative to the total weight of the dye composition.

In general, the addition salts of the oxidation bases and of the couplers that may be used in the context of the invention are chosen especially from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, ammonia, amines or alkanolamines.

The composition in accordance with the invention may also contain one or more direct dyes that may be chosen especially from nitrobenzene dyes, azo direct dyes and methine direct dyes. The dyes may be of nonionic, anionic or cationic nature.

The medium that is suitable for dyeing, also known as the dye support, generally consists of water or a mixture of water and at least one organic solvent to dissolve the compounds that would not be sufficiently water-soluble. As organic solvent, mention may be made, for example, of C₁-C₄ lower alkanols, such as ethanol and isopropanol; polyols and polyol ethers such as 2-butoxyethanol, propylene glycol, propylene glycol monomethyl ether, diethylene glycol monoethyl ether and monomethyl ether, as well as aromatic alcohols such as benzyl alcohol or phenoxyethanol, and mixtures thereof.

The solvents are preferably present in proportions of between 1% and 40% by weight approximately relative to the total weight of the dye composition, and even more preferably between 5% and 30% by weight approximately.

The dye composition in accordance with the invention can also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic or amphoteric associative polymeric thickeners, antioxidants, penetration agents, sequestering agents, fragrances, buffers, dispersants, conditioners, for instance volatile or nonvolatile, modified or unmodified silicones, film-forming agents, ceramides, preserving agents and opacifiers.

The above adjuvants are generally present in an amount for each of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between about 3 and 12 and preferably between about 5 and 11. It may be adjusted to the desired value using acidifying or basifying agents usually used in the dyeing of keratin fibers, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as mono-, di- and triethanolamine and derivatives thereof, sodium hydroxide, potassium hydroxide and the compounds of formula (II) below:

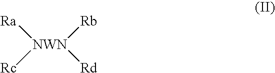

in which W is a propylene residue that is unsubstituted or substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The dye composition according to the invention may be in various forms, such as in the form of liquids, creams or gels, or in any other form that is suitable for dyeing keratin fibers, and especially human hair.

The process of the present invention is a process in which the composition according to the present invention, as defined above, is applied to the fibers, and the color is developed using an oxidizing agent. The color may be developed at acidic, neutral or alkaline pH and the oxidizing agent may be added to the composition of the invention just at the time of use, or it may be used starting with an oxidizing composition containing it, which is applied simultaneously with or sequentially to the composition of the invention.

According to one particular embodiment, the composition according to the present invention is mixed, preferably at the time of use, with a composition containing, in a medium that is suitable for dyeing, at least one oxidizing agent, this oxidizing agent being present in an amount that is sufficient to develop a coloration. The mixture obtained is then applied to the keratin fibers. After a leave-in time of 3 to 50 minutes approximately, preferably 5 to 30 minutes approximately, the keratin fibers are rinsed, washed with shampoo, rinsed again and then dried.

The oxidizing agents conventionally used for the oxidation dyeing of keratin fibers are, for example, hydrogen peroxide, urea peroxide, alkali metal bromates, persalts such as perborates and persulfates, peracids and oxidase enzymes, among which mention may be made of peroxidases, 2-electron oxidoreductases such as uricases, and 4-electron oxygenases, for instance laccases. Hydrogen peroxide is particularly preferred.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibers preferably ranges between 3 and 12 approximately and more preferably between 5 and 11. It may be adjusted to the desired value by means of acidifying or basifying agents usually used in the dyeing of keratin fibers and as defined above.

The ready-to-use composition that is finally applied to the keratin fibers may be in various forms, such as in the form of liquids, creams or gels or any other form that is suitable for dyeing keratin fibers, and especially human hair.

A subject of the invention is also a multi-compartment device or dyeing “kit”, in which a first compartment contains the dye composition defined above and a second compartment contains an oxidizing composition. This device may be equipped with a means for applying the desired mixture to the hair, such as the devices described in patent FR-2 586 913 in the name of the Applicant.

The compounds that are useful in the composition of the present invention are known compounds that may be obtained using general preparation processes known to those skilled in the art. For example, the synthetic approach shown below is described in the literature up to the intermediate (2) (J. H. P. Juffermanns, C. L; Habraken; J. Org. Chem. 1986, 51, 4656; Klebe et al.; Synthesis, 1973, 294; R. Huttel, F. Büchele; Chem. Ber.; 1955, 88, 1586). In the present case, the conversion of compound 3 into compound 2 is performed using an NH₃/EtOH mixture.

The alkylation and the amination to obtain the compounds of formula (I) according to the invention are mentioned in document DE 42 34 885.

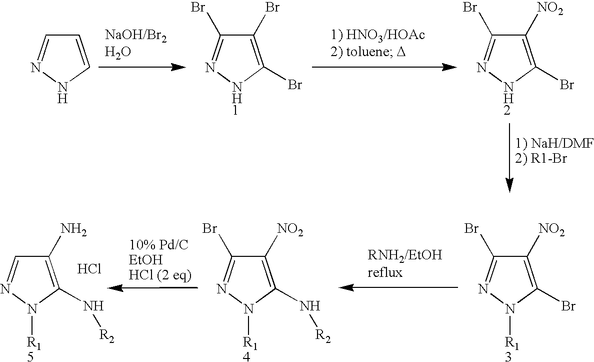

The examples that follow serve to illustrate the invention without, however, being limiting in nature.

### EXAMPLES

### Example 1

### Synthesis of 4,5-diamino-1-(2′-methoxyethyl)pyrazole dihydrochloride

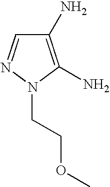

A mixture of 5-benzylamino-3-bromo-1-(2′-methoxyethyl)-4-nitropyrazole (4 g, 2.8 mmol) in ethanol (500 ml) containing a 10% Pd/C catalyst (Johnson-Mattey Type 487, dry weight 0.5 g) and 36% hydrochloric acid (0.57 g, 5.6 mmol) is hydrogenated in a Parr Autoclave (1 l) at 1 MPa for 1 hour. The catalyst is then removed by filtration and washed with ethanol, and the filtrate is evaporated under reduced pressure. A crude orange-colored solid (2.8 g) is thus obtained, and is triturated in EtOAc (20 ml) for 1 hour. The solid is then filtered off, washed with cold EtOAc (20 ml) and then dried under vacuum to give the 4,5-diamino-1-(2′-methoxyethyl)pyrazole in the form of a beige-colored solid (0.7 g, 27%).

HPLC (purity): 99.5%

m.p.: 168.1-173.0° C.

¹H NMR: (400 MHz, d⁶-DMSO): 7.34 (1H, S, NHₐᵣₒₘ), 5.18 (1H, S_{broad}, NH), 4.09 (2H, t, J=5.5 Hz, CH₂N), 3.61 (2H, t, J=5.5 Hz, CH₂O), 3.23 (3H, S, OCH₃)

### Example 2

### Dye Composition Containing 4,5-diamino-1-(2′-methoxyethyl)pyrazole dihydrochloride

The following dye composition was prepared:

| Example | 2 |
|---|---|
| 4,5-Diamino-1-(2′-methoxyethyl)- | 0.744 g |
| pyrazole dihydrochloride | |
| Pyrazolo[1,5-a]pyrid-3-ylamine | 0.508 g |
| hydrochloride | |
| 2-Methyl-5-aminophenol | 0.78 g |
| Dye support | (*) |
| Demineralized water qs | 100 g |

Dye Support

| | | |
|---|---|---|
| Benzyl alcohol | 2 | g |
| Polyethylene glycol 8 EO | 3 | g |
| Ethanol | 18 | g |
| (C8-C10)alkyl polyglucoside as an aqueous | 5 | g in this |
| solution containing 60% active material | | case |
| buffered with ammonium citrate, sold | | |
| under the name Oramix CG110 by SEPPIC | | |
| Aqueous ammonia containing 20% NH₃ | 10 | g |
| Sodium metabisulfite | 0.205 | g |
| Sequestering agent | qs | |

At the time of use, the composition is mixed with an equal weight of 20-volumes aqueous hydrogen peroxide solution (6% by weight).

The mixture obtained is applied to locks of natural or permanent-waved gray hair containing 90% white hairs, at a rate of 10 g per 1 g of hair. After a leave-in time of 30 minutes, the locks are rinsed, washed with a standard shampoo, rinsed again and then dried.

The locks are assessed visually. The glint on the lock is a strong coppery red.

1-19. (Canceled)

20. A dye composition comprising, in a suitable dyeing medium,
at least one first oxidation base chosen from 4,5-diaminopyrazole oxidation bases of formula (I) and addition salts thereof: 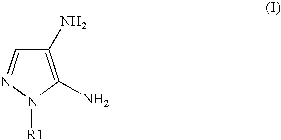
wherein R₁ is chosen from C₁-C₆ alkyl radicals substituted with at least one OR radical, wherein R is chosen from C₁-C₆ alkyl radicals;
at least one second oxidation base chosen from heterocyclic oxidation bases and addition salts thereof; and
at least one coupler.

21. The composition according to claim 20, wherein the at least one second oxidation base is chosen from pyridine, pyrimidine, and pyrazole heterocyclic oxidation bases, and addition salts thereof.

22. The composition according to claim 21, wherein the at least one second oxidation base is chosen from 3-aminopyrazolo[1,5-a]pyridine oxidation bases and addition salts thereof.

23. The composition according to claim 22, wherein the 3-aminopyrazolo[1,5-a]pyridine oxidation base is pyrazolo[1,5-a]pyrid-3-ylamine.

24. The composition according to claim 20, wherein the at least one coupler is chosen from meta-phenylenediamines, meta-aminophenols, meta-diphenols, naphthalene-based couplers, heterocyclic couplers, and addition salts thereof.

25. The composition according to claim 24, wherein the at least one coupler is chosen from meta-aminophenols.

26. The composition according to claim 20, wherein, in formula (I), R₁ is chosen from C₁-C₄ alkyl radicals substituted with at least one radical OR, wherein R is chosen from C₁-C₄ alkyl radicals.

27. The composition according to claim 20, wherein the at least one first oxidation base is 4,5-diamino-1-(2′-methoxyethyl)pyrazole.

28. The composition according to claim 20, further comprising at least one additional oxidation base chosen from para-phenylenediamines, bis(phenyl)alkylenediamines, para-aminophenols, ortho-aminophenols, and addition salts thereof.

29. The composition according to claim 20, wherein the at least one first oxidation base and the at least one second oxidation base are each present in an amount ranging from 0.001% to 10% by weight, relative to the total weight of the composition.

30. The composition according to claim 29, wherein the at least one first oxidation base and the at least one second oxidation base are each present in an amount ranging from 0.005% to 6% by weight, relative to the total weight of the composition.

31. The composition according to claim 20, wherein the at least one coupler is present in an amount ranging from 0.001% to 10% by weight, relative to the total weight of the composition.

32. The composition according to claim 20, further comprising at least one oxidizing agent.

33. The composition according to claim 32, wherein the at least one oxidizing agent is chosen from hydrogen peroxide, urea peroxide, alkali metal bromates, persalts, peracids, and oxidase enzymes.

34. The composition according to claim 20, further comprising at least one direct dye.

35. A process for the oxidation dyeing of keratin fibers comprising,
applying to the fibers at least one dye composition comprising, in a suitable dyeing medium,
at least one first oxidation base chosen from 4,5-diaminopyrazole oxidation bases of formula (I) and addition salts thereof: 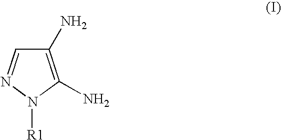
wherein R₁ is chosen from C₁-C₆ alkyl radicals substituted with at least one OR radical, wherein R is chosen from C₁-C₆ alkyl radicals;
at least one second oxidation base chosen from heterocyclic oxidation bases and addition salts thereof; and
at least one coupler; and
developing a color with at least one oxidizing agent.

36. The process according to claim 35, wherein at the at least one oxidizing agent is chosen from hydrogen peroxide, urea peroxide, alkali metal bromates, persalts, peracids, and oxidase enzymes.

37. The process according to claim 35, wherein the at least one oxidizing agent is mixed at the time of application with the at least one dye composition.

38. The process according to claim 35, wherein the at least one oxidizing agent is applied to the fibers in the form of at least one oxidizing composition simultaneously with or sequentially to the at least one dye composition.

39. A multi-compartment dyeing kit comprising
(a) at least one first compartment comprising at least one dye composition comprising, in a suitable dyeing medium,
at least one first oxidation base chosen from 4,5-diaminopyrazole oxidation bases of formula (I) and addition salts thereof: 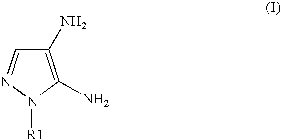
wherein, R₁ is chosen from C₁-C₆ alkyl radicals substituted with at least one OR radical, wherein R is chosen from C₁-C₆ alkyl radicals;
at least one second oxidation base chosen from heterocyclic oxidation bases and addition salts thereof; and
at least one coupler and
(b) at least one second compartment comprising at least one oxidizing composition.

40. A method for dyeing keratin fibers comprising applying to the fibers at least one dye composition comprising, in a suitable dyeing medium,
at least one first oxidation base chosen from 4,5-diaminopyrazole oxidation bases of formula (I) and addition salts thereof: 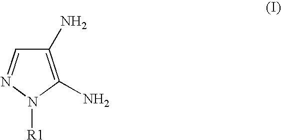
wherein R₁ is chosen from C₁-C₆ alkyl radicals substituted with at least one OR radical, wherein R is chosen from C₁-C₆ alkyl radicals;
at least one second oxidation base chosen from heterocyclic oxidation bases and addition salts thereof; and
at least one coupler.
